(19) United States
(12) Patent Application Publication
(10) Pub. No.: US 2005/0267326 A1
(43) Pub. Date: Dec. 1, 2005

(54) Percutaneous chemical sensor based on fluorescence resonant energy transfer (FRET)

(75) Inventors: Gerald E. Loeb, South Pasadena, CA (US); Kuo-Chih (Vincent) Liao, Los Angeles, CA (US); Thieo Hogen-Esch, Los Angeles, CA (US)

Correspondence Address: MCDERMOTT WILL & EMERY LLP, Suite 3400, 2049 Century Park East, Los Angeles, CA, 90067, US

(73) Assignee: Alfred E. Mann Institute for Biomedical Eng. at the University of Southern California

(21) Appl. No.: 11/088,460

(22) Filed: Mar. 24, 2005

Related U.S. Application Data
(63) Continuation-in-part of application No. 10/263,272, filed on Oct. 2, 2002.
(60) Provisional application No. 60/326,908, filed on Oct. 2, 2001.
(60) Provisional application No. 60/556,563, filed on Mar. 25, 2004.
(60) Provisional application No. 60/651,318, filed on Feb. 9, 2005.

(51) Int. Cl. A61B 1/00; C12M 1/34

(52) U.S. Cl. 600/102; 435/287.2

(57) ABSTRACT

A biosensing device for detecting biological analytes, and methods of use and manufacture, are disclosed. The device includes a biosensing element that can remain implanted for extended periods of time. The biosensing element is connected to an optical fiber terminating outside of the body. The optical fiber is also connected to an information analyzer. The information analyzer directs light through the optical fiber into the biosensing element. The light excites fluorophores, created by a chemical reaction between analytes and biosensing material within the biosensing element. Emitted fluorescent light is redirected through the optical fiber to the information analyzer. Detectors detect the deflected fluorescent emissions and, according to their determined wavelength, report the presence or quantity of specific analytes to the patient on an external display.

### CROSS REFERENCE OF RELATED APPLICATIONS

This application is a continuation-in-part of copending U.S. application Ser. No. 10/263,272, filed Oct. 2, 2002, entitled “Internal Biochemical Sensing Device,” the contents of which are incorporated herein by reference. This application is related to U.S. Provisional Application Ser. No. 60/326,908, filed Oct. 2, 2001, entitled “Percutaneous Photochemical Sensing Device and Method of Manufacture”, which is incorporated herein by reference. This application is also related to and claims the benefit of the filing dates of U.S. Provisional Application Ser. No. 60/556,563, filed Mar. 25, 2004, entitled “Percutaneous Chemical Sensor Based on Fluorescence Resonant Energy Transfer (FRET)”; and U.S. Provisional Application Ser. No. 60/651,318, filed Feb. 9, 2005, entitled “Internal Biochemical Sensing Device”, the contents of each of which are incorporated herein by reference.

### BACKGROUND OF THE INVENTION

1. Field of the Invention

The present invention relates generally to implanted devices and methods for repeated detection of biochemical analytes.

2. General Background and State of the Art

In order to detect or manage certain diseases or conditions, it is useful to make frequent measurements of specific biochemical analytes, hereinafter referred to as “analytes,” within a patient's body over an extended period of time. For example, glucose levels in a patient's body can be monitored to guide the dosage of insulin required to treat diabetes mellitus. Another example would be monitoring the tissue concentration of therapeutic drugs such as anticoagulants, immunosuppressive agents and anticancer drugs, all of which can lead to serious complications if the tissue levels are too high or too low. Monitoring the presence and levels of such analytes in a patient's body is often a cumbersome process, making it difficult to accomplish over extended periods of time. For example, glucose monitoring is frequently performed through invasive means utilizing external glucose meters. Typically, glucose measurements are taken by pricking a patient's finger, extracting a drop of blood, and applying the blood to a test strip containing chemicals that are sensitive to the glucose in the blood sample. An optical meter is then used to analyze the blood sample on the test strip and provide the patient with a numerical glucose reading. Because readings show only a “snap shot” of blood glucose levels, repeated painful finger pricks are required over time. Also patients must carry supplies to take repeated measurements. These factors lead to patient non-compliance.

Less invasive methods for detecting analytes in a patient's body are known and practiced, but have limited effectiveness for other reasons. For example, certain transcutaneous optical absorption techniques for quantification of glucose can be based on selective absorption of light by the glucose molecule. However, such in vivo measurements are susceptible to inaccuracies due to differences in skin pigmentation, hydration, blood flow, probe placement and probe pressure. Because skin is a highly scattering medium, optical measurements taken through the skin are adversely affected by attenuation and low signal-to-noise ratio.

Thus, there is a need for a minimally invasive device and method for repeated detection of a broad range analytes from patients. There is also a need for a compact and portable, yet accurate system for detection.

### SUMMARY

In one aspect of the biosensing devices and systems, a device for detecting an analyte from within a patient's body comprises an optical fiber configured to intermittently connect and disconnect to an analyzer; and a biosensing material attached to the optical fiber comprising a polymer matrix, at least one receptor molecule attached to the polymer matrix and labeled with a first detector molecule, and at least one competitive binding molecule attached to the polymer matrix and labeled with a second detector molecule. In another aspect of the biosensing devices and systems, the concentrations of the first and second detector molecules may be low enough to minimize random proximity during FRET quenching, yet high enough to be detected by the analyzer.

In another aspect of the biosensing devices and systems, a system for detecting an analyte from within a patient's body comprises an optical fiber; a biosensing material attached to the optical fiber comprising a polymer matrix, at least one receptor molecule attached to the polymer matrix and labeled with a first detector molecule, and at least one competitive binding molecule attached to the polymer matrix and labeled with a second detector molecule; and an analyzer that is configured to rapidly and intermittently connect and disconnect to the optical fiber, and that is configured to emit light into the optical fiber, receive light from the biosensing material, and process information from the received light.

In still a further aspect of the biosensing devices and systems, a method of manufacturing an implantable biosensing device comprises modifying the surface of a first end of an optical fiber to create an adhesion region; submerging the first end of the optical fiber into a matrix precursor solution; delivering ultraviolet light through a second end of the optical fiber; and removing the first end of the optical fiber from the matrix precursor solution.

In yet another aspect of the biosensing devices and systems, a method of detecting an analyte from within a patient's body comprises implanting an optical fiber having an implanted and free end within the patient's body such that the implanted end lies within the percutaneous region and the free end protrudes from the patient's body; allowing the implanted end of the optical fiber to remain in the percutaneous region of the body without removal for at least seven days; allowing the free end of the optical fiber to remain unconnected to any device for a substantial portion of the at least seven days while the implanted end remains in the percutaenous region of the body; connecting and disconnecting the free end to a measuring instrument; and testing the analyte with the measuring instrument while it is connected to the free end.

One advantage of various embodiments of the biosensing devices, methods and systems is that problems associated with previous methods of repeatedly measuring patient analytes are avoided. For example, optical fibers are small, thin, lightweight, chemically stable and generally biocompatible, allowing them to be relatively easily inserted into a patient's body and maintained for repeated measures over time. Combined with fluorescence techniques for analyte detection, changes in fluorescence intensity and/or wavelength caused by binding of the analyte with a biosensing material, an optical fiber can transmit fluorescing evidence of the analyte from within the patient's body to an external analyzer.

It is understood that other embodiments of the biosensing devices and systems will become readily apparent to those skilled in the art from the following detailed description, wherein it is shown and described only exemplary embodiments of the biosensing devices, methods and systems by way of illustration. As will be realized, the biosensing devices, systems and systems are capable of other and different embodiments and its several details are capable of modification in various other respects, all without departing from the spirit and scope of the biosensing devices, methods and systems. Accordingly, the drawings and detailed description are to be regarded as illustrative in nature and not as restrictive.

### BRIEF DESCRIPTION OF THE DRAWINGS

Aspects of the biosensing devices and systems are illustrated by way of example, and not by way of limitation, in the accompanying drawings, wherein:

FIG. 1 is a schematic illustration of an exemplary biochemical sensing system;

FIG. 2 illustrates an exemplary embodiment of a biosensing element implanted in a patient;

FIG. 3 is a schematic illustration of an exemplary analyzer and biosensing device.

### DETAILED DESCRIPTION

The detailed description set forth below in connection with the appended drawings is intended as a description of exemplary embodiments and is not intended to represent the only embodiments in which the biosensing devices, methods and systems can be practiced. The term “exemplary” used throughout this description means “serving as an example, instance, or illustration,” and should not necessarily be construed as preferred or advantageous over other embodiments. The detailed description includes specific details for the purpose of providing a thorough understanding of the biosensing devices, methods and systems. However, it will be apparent to those skilled in the art that the biosensing devices, methods and systems may be practiced without these specific details.

In an exemplary embodiment, minimally invasive biosensors are attached to the ends of percutaneously injected optical fibers. The fiber-optic biosensor takes advantage of the configuration of chronically implanted artificial hair used for cosmetic purposes. Such hairs consist of filaments of synthetic polymer that can be injected into the scalp, where they form a stable epithelial interface. Likewise, the biosensor is implantable underneath the skin into a well-vascularized subcutaneous space such as the scalp. In an exemplary embodiment, a single optical fiber makes up the “shaft” of the hair, and the sensing system is the “follicle”.

In order to manage certain diseases, it is often beneficial to make frequent measurements of specific biochemicals over an extended period of time. Accordingly, some embodiments of the biosensing devices and systems can be used to measure glucose. Other analytes that can be analyzed by embodiments include, but are not limited to, hormones related to fertility, premature delivery and other late-term complications of pregnancy such as eclampsia. Some embodiments of the technology could be applied to assay tissue levels of drugs that have narrow margins between effective and dangerous levels, such as cytotoxic chemotherapeutics (e.g. Taxol) and anticoagulants. Clinically significant analytes that can be analyzed include, but are not limited to: glucose, cholesterol, amylase, urea, triglycerides, pH, Creatinine kinase, Creatinine, Aspartate aminotransferase, Phenylalanine, Lactate dehydrogenase, Akaline phosphotase, GOT, Bilirubin, oxygen, carbon dioxide, ammonia, Theophylline, Dilantin, Gentamicin, Tobramicin, Digoxin, Coumadin, Vincristine, cortisol, estriol, progesterone, aldosterone, cortisone, thyroxine binding globulin, placental lactogen, prolactin, human chorionic gonadotropin, insulin, parathyroid hormone, growth hormone, angiotensin, oxytocin, vasopressin, IgM (total), IgG (specific), Syphilis, Rubella, Hepatitis, Alpha-fetoprotein, and various cancer proteins.

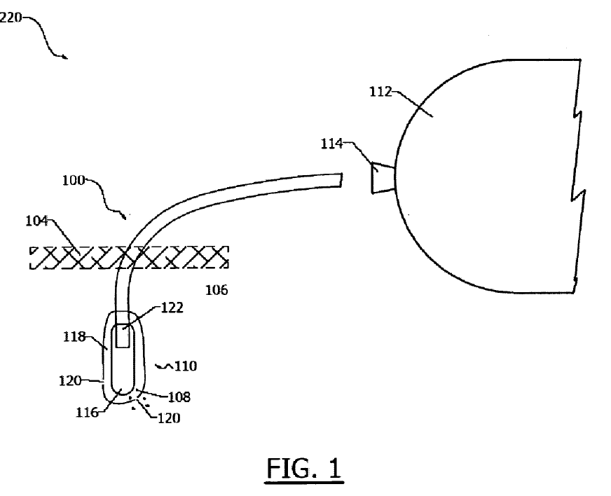

FIG. 1 illustrates am exemplary compact and portable biosensing system 220 comprising a biosensing device 100, an analyzer 112, and an exemplary mode of positioning relative to a patient's body. The exemplary biosensing device comprises an optical fiber 102 that extends through the patient's skin 104. The optical fiber 102 may be injected percutaneously to sample interstitial fluid (e.g. in the scalp or forearm), or in any other region in which analytes 108 are being tested. The biosensing device 100 includes a biosensor element 110, attached to a first end of the fiber 102 that is inserted into the patient's body. The second, opposite, end of the fiber 102 is releasably attached to an analyzer 112 by means of a connector 114. The analyzer 112 receives light emitted by the biosensing element 110 via the optical fiber 102, then filters and analyzes the received light to detect the presence and/or quantity of analytes within the patient's body.

In an exemplary embodiment, the analyzer 112 is sized and configured to be easily carried by the patient. The information analyzer 112 is portable such that it may be easily moved or even worn by the patient, sized and configured to be easily carried by the patient. For example, the information analyzer 112 could be sized to fit within a patient's hand, and could be light enough to be easily moved by the patient, or attached to the patient's clothing or to a strap that is worn by the patient. Because of its portability and small size, the information analyzer 112 may be used to take continuous measurements, such as when the patient wears it on his body or clothing. Its small size also makes the information analyzer 112 convenient for taking frequent, yet intermittent measurements, such as when the patient wears it or simply carries it with him because it is easily portable and accessible. In use, the patient slips the free external end of the optical fiber 100 of the implanted biosensing device into a connector 114, which triggers the analyzer 112 to take a reading and display the results to the user. In some exemplary embodiments, the implanted device can remain continuously in the patient without removal for varying lengths of time. For example, in one exemplary embodiment, the implanted device can remain continuously in the patient without removal for at least one day. In another exemplary embodiment, the implanted device can remain continuously in the patient without removal for at least seven days. In a further exemplary embodiment, the device can remain continuously in the patient without removal for at least one month.

The information transmitted through the optical fiber 102 is light energy (photons at different wavelengths), and the connector is an optical connector 114, to ensure the presence of an optical connection between the optical fiber 102 and the analyzer 112. In this exemplary embodiment, the analyzer 112 exposes the biosensor element 110 to excitation light of a first wavelength from light emitting diode (LED) that is directed through an optical connector 114 to optical fiber 102 to the biosensor element 110, and in response receives emitted fluorescent light of at least a second wavelength from the biosensor element, directed through the optical fiber in the opposite direction. The emitted fluorescent light can then be filtered and measured by the analyzer 112 to identify and/or quantify the analytes detected by the biosensor element 110. The analyzer 112 may identify the presence of specific analytes by measuring the wavelength of the fluorescent light emitted, and may measure the quantity of analytes present by measuring the intensity of the fluorescent light emitted.

In one exemplary embodiment, the biosensor element 110 comprises biosensing material 116 located substantially at the end of the optical fiber 102. In some embodiments, it may be desirable to prevent substantially direct contact between the biosensing material 116 and patient tissue 106. In such cases, the biosensor element 110 may include a containment matrix 118 that substantially contains the biosensing material 116 within a reaction region that is in close proximity to the end of the optical fiber 102. In some embodiments, for example, the containment matrix may comprise polyethylene glycol (PEG), a silicone-based material, or other biocompatible material known to those skilled in the art. Further, the containment matrix 118 may be configured to be in contact with or form a seal with the optical fiber 102. The containment matrix 118 thereby can contain the biosensing material so that it does not diffuse away from the biosensor element. The containment matrix 118 may also contain the products of a reaction between analytes 108 and the biosensing material 116. This containment of the reactive products can prevent them from dispersing throughout the patient's body such that they are retained within a concentrated area for signal communication to the optical fiber 102. The containment matrix 118 can include pores 120 to allow analytes 108 to diffuse within the containment matrix 118 to contact the biosensing material. The pores 120 may be inherently formed due to the characteristics of the material used for the containment matrix 118 or, if the selected material is not sufficiently porous, then pores may be explicitly created therein, for example by burning holes using a tightly focused laser beam such as an excimer laser. The pores can be sized such that they are large enough to allow the diffusion of analytes 108 into the reaction region, and small enough to prohibit the passage of other elements from the reactive region to other areas of the patient's body.

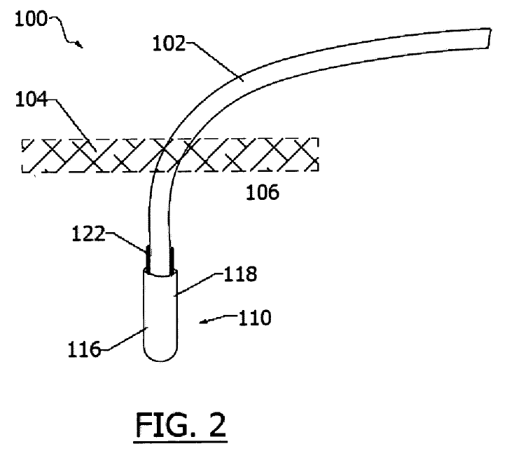

FIG. 2 illustrates another exemplary embodiment of the biosensor element 110. In the embodiment illustrated in FIG. 2, the containment matrix 118 and biosensing material 116 can be combined. The materials of the containment matrix 118 can be selected to be biocompatible with the patient, permeable to the analytes being detected, capable of chemically or physically trapping the biosensing material 116 (including its fluorophores) and of a material that forms a strong adhesion to the optical fiber 102. The containment matrix can be attached directly to the internal end of the optical fiber, permitting efficient and constant coupling to a small sensing structure. In an exemplary embodiment, polyethylene glycol (PEG) polymers can be used since PEG demonstrates good biocompatibility and structural integrity. The polymer can be applied to the optical fiber in an unpolymerized state, and then polymerized to enhance stability of the structure by gamma irradiation, chemical cross-linking or UV radiation.

An exemplary method of preparing a containment matrix precursor solution combines a PEG carrier with tetramethylrhodamine isothiocyanate (TRITC-dextran), fluorescein isothiocyanate concanavalin A (FITC-Con A), and fluorophores. One method is described by Russell et al. (R. J. Russell, M. V. Pishko, C. C. Gefrides, M. J. McShane and G. L. Cote, 1999, “A Fluorescence-Based Glucose Biosensor Using Concanavalin A and Dextran Encapsulated in a Poly(ethylene glycol) Hydrogel”, Anal. Chem 71:3126-3132), and is hereby incorporated by reference. For example, FITC-Con A and TRITC-dextran are dissolved prior to use in about 0.1 M PBS (about pH 7.4). The FITC-Con A solution and PEG-NHS, polyethylene glycol-N-hydroxysuccinimide (Con A/PEG-NHS=100 μL/1 mg) are added to PEG-DA, polyethylene glycol-diacrylate (for example, the volume ratio of PEG-DA to fluorescein solution can be 2:1) and the resultant mixture can be vortexed for approximately 30 minutes. TRITC-dextran, 100 μL of TPT, and 10 mg DMPA are added and vortexed for approximately 30 minutes.

In an exemplary embodiment, the containment matrix is attached to the optical fiber by dipping the optical fiber into a containment matrix precursor solution, such as the solution described above. UV light (for example, 4 W/cm²) can then be passed through the fiber to induce cross-linking polymerization onto the end of the fiber. After the fiber is pulled out from the solution, the fiber can be dipped again, removed from the solution, and polymerized with UV from the side to increase the interface contact area for better adhesion.

In some embodiments, the optical fiber 102 may be composed of a number of different materials such as, for example, glass, silicon or plastic. For example, glass has desirable optical properties and can be configured to have a silicon outer surface that can be modified to bind different coatings. Some embodiments can be covered with a variety of biocompatible polymers that enhance the fiber optics' strength and tissue integration. Although the optical fiber 102 does not have a specific size requirement, fibers having a diameter between about 50 μm and about 200 μm can be used for ease of insertion through the skin 104 of a patient. Fibers within this range of sizes are also sufficiently large for effective data transmission, suitably flexible that a patient can manipulate them with ease, and sufficiently strong to withstand patient wear. For example, a 100 μm/110 μm (core/cladding) glass fiber can be bent to a radius of about 0.5 mm before fracturing.

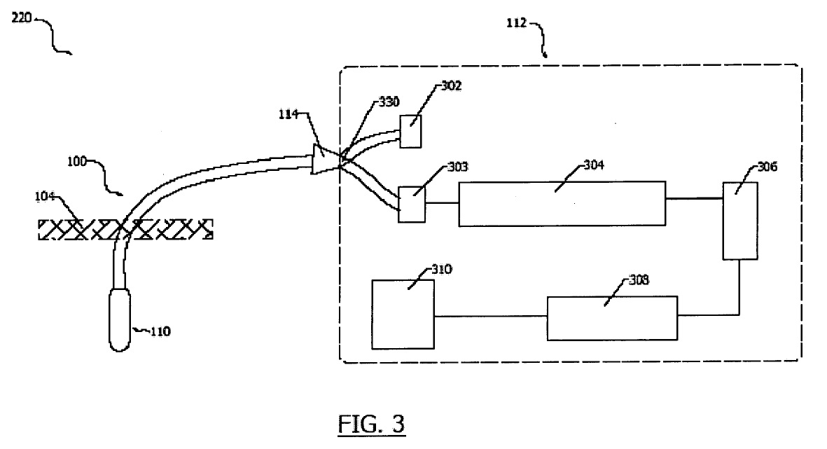

FIG. 3 is a diagram of an exemplary analyzer 112, which is sized and configured as a pen-like, battery-powered device with LCD read-out. In the exemplary embodiment, the analyzer 112 comprises a photonic analyzer. Specifically, the information analyzer comprises a fluorescence spectrophotometer that photonically excites a sample within, or in proximity to the biosensor element 110, and then detects the wavelength and/or intensity of any optical signal emitted there from. In some embodiments, the analyzer 112 comprises a light source 302, optical connector 114, optical splitter 330, one or more optical filters 304, lens coupler 303, a photon detector 306, signal processing electronics 308 and a patient readout system 310. In some embodiments, the optical splitter 330 can include fused fiber optical couplers, half-silvered mirrors, dichroic mirrors, and diffused optical waveguides.

In an exemplary method employed by the analyzer 112, an excitation wavelength is produced by light source 302. The light source 302 may be, for example, a fiber-coupled blue laser diode with a built-in source driver capable of producing, for example, 20 mW-24 mW. Alternatively, blue light-emitting diodes (LED) with high output power may be used as the light source 302. Those skilled in the art will also recognize other suitable excitation light sources such as a broadband, incandescent light source from which a tunable, narrow band of excitation wavelengths can be selected by a diffraction grating or prism.

In an exemplary embodiment, the filtering member 304 (which may also be an optical fiber) includes an acoustic tunable filter region. Filtering members that can be used and/or adapted to be used in some embodiments are described in U.S. Pat. No. 5,611,004 (Chang) and by Birk et al. (Birk, T A, Russel, P S J, Pannel, C N (1994) “Low power acoustic-optical device based on a tapered single-mode fiber.” IEEE photon. Technol. Lett. 6: 725-727), the contents of each of which are incorporated by reference herein. As fluorescent emissions from the fluorophore pass through the filter section, a PZT transducer deflects photons with wavelengths matched to the acoustic wavelength into detector, where they are captured and quantified by the photodiode. The electronic feedback control of the filter band can be used advantageously to identify and quantify the two fluorescence peaks even if the accuracy of the filter drifts over time. An algorithm in the power and signal processing unit 308 can sweep the center wavelength of the filter over a range of wavelengths while measuring the output of photodetector 306. The location of fluorescence peaks can be identified by a change in the slope of the fluorescence intensity from positive to negative as a peak is traversed. Photon counts on either side of the peak can be integrated to improve the signal to noise ratio. Other potentially useful algorithms for digital signal processing can be used by those with skill in the art.

In an exemplary embodiment, adhesion between the containment matrix and the optical fiber can be achieved and/or enhanced in numerous ways in order to prevent these two components from physically separating. For example, mild etching at adhesion region 122, illustrated in FIGS. 1 and 2, can be used to increase surface roughness of the glass fiber by immersing it in hydrofluoric acid (for example, 25% hydrofluoric acid for 10 minutes). A portion of the etched fiber can then be cleaved off to create a clean end to minimize scattering of light into and out of the end of the fiber that would occur at an etched surface. In some embodiments, a portion of the etched fiber can be beveled at an angle. In another exemplary embodiment, chemical agents such as (aminopropyl) triethoxysilane can modify the fiber surface and provide covalent bonding with the matrix after polymerization to enhance the containment matrix adhesion at adhesion region 122. In an alternative exemplary embodiment, mechanical abrasion can increase the surface roughness of optical fiber 102. The surface roughness modification should avoid damage to the optical properties of the cladding. The limiting factor of all of the above methods appears to be the surface area of the optical fiber actually in contact with the matrix. This can be increased by using multiple dip coats and photopolymerization steps, which builds up a matrix with a larger volume (increasing the amount of dye available to fluoresce) and increases the surface area of the containment matrix 118 in contact with region 122.

An exemplary embodiment of the biosensing device detects the presence of analytes within the patient's tissues by employing a biosensing material 116. A chemical binding or reaction between the analyte 108 and the biosensing material 116 can give rise to a state change that can be transmitted to and detected by the information analyzer 112. The biosensing material 116 takes advantage of the unique specificity of biosensing molecules for analyte(s) of interest. This high selectivity allows the analyte to be measured even when mixed with other substances, such as occurs in blood or extracellular fluids. The biosensor materials can be selected to maintain mechanical stability and biocompatibility during chronic implantation.

In an exemplary embodiment, fluorescence optical sensing can be utilized. The biosensing material includes molecules that undergo a change in fluorescent emission in proportion to the concentration of analyte of interest in the surrounding medium. In some embodiments, many different fluorescent dyes can be bound covalently to molecules that bind specifically to analytes (such as glucose). For example, some fluorescent molecules that may be used are described in publications by Tompson, McNichols et al., and Czarnik (Thompson, R. B. “Fluorescence-Based Fiber-Optic Sensors.” Topics in Fluorescence Spectroscopy, Vol. 2: Principles. New York: Plenum Press 1991: 345-65; McNichols R and Cote G. “Optical glucose sensing in biological fluids: an overview.” Journal of Biomedical Optics January 2000, 5:5-16; Czarnik, A. (1993) Fluorescent Chemosensors for Ion and Molecule Recognition. Washington: American Chemical Society), each of which are herein incorporated by reference. Some embodiments of the biosensing devices and systems may use other optical sensing techniques such as absorption and transmission, which are well known to individuals skilled in the art.

Exemplary embodiments of the biosensing devices and systems can utilize various potential fluorescence sources. For example, two particular alternative systems may be useful where fluorescence is selected as the mode of optical transmission, as described by Krohn (Krohn, D. Fiber Optic Sensors: Fundamentals and Applications. North Carolina: Instrument Society of America, 1988), which is incorporated herein by reference. In one system, the analyte itself is fluorescent. In another system, the analyte is not fluorescent but interacts with a fluorophore that emits a fluorescent signal. Where the analyte to be detected is glucose, a number of techniques may be employed, including, but not limited to enzyme based and competitive affinity binding. See, for example, McNichols R and Cote G. “Optical glucose sensing in biological fluids: an overview.” Journal of Biomedical Optics January 2000, 5:5-16, incorporated herein by reference.

In an exemplary embodiment having analytes that do not emit fluorescence, the combination of FRET and a specific receptor-analyte competition model can be used as a photonic assay method for an implantable sensor that is likely to be slowly biodegrading. In such embodiments, quantitative measurements may depend on the ratio of fluorescence at two wavelengths.

Another exemplary embodiment of the biosensing material and system utilizes fluorescence resonance energy transfer (FRET) in a receptor-analyte competition assay. FRET depends on the proximity of two fluorophores; if the distance between them is less than the Forster radius, energy absorbed by the first fluorophore is transferred efficiently to the second fluorophore, which then emits at a longer wavelength. The externally detectable fluorescence associated with the short wavelength fluorophore is thus decreased or “quenched”; the long wavelength fluorescence actually increases. In some embodiments quantum dots, which can generate narrow band (for example, 470 nm) emissions suitable for exciting a second fluorophore and can be excited with light source having much shorter wavelength, could replace the traditional fluorescence photodonor. This combination may produce more efficient and more readily detectable FRET. For example, if a receptor, which binds the target analyte, is labeled with one type of fluorophore, and a competitive ligand of the target analyte is labeled with the other dye, the affinity between receptor and the competitive ligand brings the two dyes in proximity and results in FRET quenching. When an analyte approaches the receptor, it replaces the ligand and reverses the quenching phenomenon, and the quantity of the analyte can be measured by the change in quenching.

An exemplary embodiment of the biosensor uses an affinity-binding assay for polysaccharides based on the jack bean lectin concanavalin A (ConA), as described by Mansouri et al (Mansouri S, Schultz J. “A Miniature Optical Glucose Sensor Based on Affinity Binding.” Biotechnology 1984, 885-90), which is incorporated herein by reference. Dextran binds to ConA but can be displaced by glucose. Dextran (for example, 102 kD) can be coupled to fluorescein isothiocyanate (FITC), which fluoresces at about 520 nm when excited at about 488 nm. ConA (for example, 2000 kD) can also be coupled to tetramethylrhodamine isothiocyanate (TRITC), which fluoresces at about 580 nm and can be excited at about 520 nm (the emission wavelength of FITC) as described by Meadows et al. (Meadows D and Shultz J. “Design, manufacture and characterization of an optical fiber glucose affinity sensor based on an homogeneous fluorescence energy transfer assay system.” Analytica Chimica Acta January 1993, 280:21-30), which is incorporated by reference. The TRITC-ConA and FITC-Dextran can be incorporated into PEG spheres (as described by Russell et al. Russel R; Pishko M; Gefrides C and Cote G. “A fluorescent glucose assay using poly-I-lysine and calcium alginate microencapsulated TRITC-succinyl-Concanavalin A and FITC-dextran.” IEEE Engineering in Medicine and Biology 1998, 20:2858-61; hereby incorporated by reference), where they have sufficient mobility to bind and result in FRET between them.

In some embodiments, the size of both receptor and competitive ligand, and the position of dye-labeling site and analyte-binding site on the receptor are chosen to optimize the efficiency of FRET. The efficiency of FRET is R₀⁶/(R₀⁶+R⁶), which R is the distance between the two fluorophores. The value of Forster radius (R₀) depends on the extinction coefficients, quantum yields, and mutual orientation of the two specific dyes and solvent environment. In some embodiments, the size of both receptor and ligand should not be much larger than Forster radius. In some embodiments of the affinity-binding model mentioned above, the amount of quenching achievable for the large molecular weight dextran (with molecular weight of about 155 kD, dye labeling ratio of about 2 moles dye/mole, and a radius of about 85 angstroms) is less than for the smaller dextran (with molecular weight of about 3 kD, dye labeling ratio of 1 mole dye/mole, and a radius about 14 angstroms).

In an exemplary embodiment, concentration can also influence the distance (R) of two fluorophores. FRET quenching can be triggered by affinity, which typically occurs when concentrations of both the labeled receptor and the labeled ligand are low enough to minimize random proximity. In other embodiments, the concentrations of both fluorescence labeled materials can be high enough to reach the sensitivity limit of the photodetector in the analyzer. The working range of the two fluorophores can be defined by the two concentration limitations.

The affinity between ligands and receptors can be reduced to a low enough level so that the target analytes can efficiently compete to interact with the binding site. Typically, the concentration of target analytes is located in the range of nM-pM in normal physiological conditions. In an exemplary embodiment of the affinity-binding model, using betacyclodextrin instead of linear dextran reduces the affinity (because of its rigid circular structure) between this saccharide and Con A. This permits higher concentrations (in some embodiments, at least 10 fold) of the fluorescent analytes to be used while preserving sensitivity to physiological concentrations of glucose.

In an exemplary embodiment, receptors, antibodies, and enzymes that specifically interact with the analyte(s) to be detected may be immobilized by physical capture within or covalent bonding to a biocompatible, polymeric matrix such as can be formed by the polymerization of various analogues of ethylene oxides to form, for example, polyethylene glycol. In one exemplary embodiment of the glucose biosensing material 116, the FITC-concanavalin-A is covalently bound to a polyethylene glycol that contains an N-hydroxysuccinimide ester group. The TRITC-dextran can be trapped within the small pores of the dense polyethylene glycol polymer, which is formed when polyethylene glycol diacrylate (with, for example, molecular weight of abouit 575 daltons) is illuminated with ultraviolet light. In an exemplary embodiment of the biosensing material, a PEG carrier can serve as a polymer matrix, FITC-Con A molecules attached to the PEG can act as a labeled receptor, and TRITC-dextran connected to the PEG can serve as a competitive binding molecule that competes with the patient's glucose to bind with the FITC-Con A receptor.

In another exemplary embodiment, the labeled betacyclodextrin can be modified with acryloyl group, which will provide a covalent binding site for PEG matrix, the same functional group used for the UV polymerization. A solution of acryloyl chloride (about 0.54 g, 6 mmole) in about 10 ml CH₂Cl₂ is added dropwise to a solution of TARMA-ABCD (about 3 mmole) and triethylamine (about 3.2 g, 31.7 mmole) in about 60 ml CH₂Cl₂ at −5 C during approximately one hour. The reaction mixture is stirred over night at room temperature, and then triethylamine hydrochloride is filtered off. The filtrate is diluted with about 100 ml CH₂Cl₂ and extracted with about 2×50 mL NaHCO₃ (10%) and about 1×50 mL brine. The organic phase is dried over MgSO4, filtered and distilled to give crude product. (Sha). The effectiveness of the binding can be assayed by measuring the fluorescence of the supernatant after prolonged soaking of polymerized matrix material in saline.

Other exemplary embodiments of the biosensor can use quantum dot fluorophors. One of the technical challenges in optical biosensors is to filter out the relatively intense excitation wavelength from the two fluorescence wavelengths. The excitation light tends to backscatter from the optical connector, the junction between the optical fiber, the splitter, and the optical fiber in the portable measurement instrument, and the polymer matrix on the internal end of the optic fiber. The larger the differences in wavelength, the easier it is to achieve adequate filtering to avoid saturating the fluorescence detection circuitry and resolve the two peaks whose ratio are measured. Quantum dots, or fluorescent semiconductor nanocrystals, are inorganic spheres with nanometer dimensions that can be excited with a broad range of short wavelengths and produce high efficiency fluorescence at longer wavelengths that are precisely controllable. Quantum dots are described by Michalet et al. (Michalet et al., Quantum dots for live cells, in vivo imaging, and diagnostics, Science, Jan. 28, 2005; 307(5709):538-44), which is hereby incorporated by reference. In an exemplary embodiment, a conventional fluorophor with a narrow band of excitation wavelength can be conjugated to one of the reactants (e.g. TRITC to Concanavalin) while one or more quantum dots that emit the wavelength that excites the conventional fluorophore can be conjugated to the other reactant (e.g. dextran). A relatively short wavelength can be used to excite the quantum dots and their fluorescence will be absorbed by the TRITC and reemitted at a much longer wavelength when the two fluorophors are within the Forster radius.

Another exemplary application of the biosensor is on chemotherapeutics, such as such as taxol, which bind to the intracellular protein tubulin. The affinity between tubulin and taxol provides the basis for taxol detection. In one embodiment, taxol can be labeled with FITC, and tubulin can be conjugated to a quantum dot, which can generate about a 470 nm emission when excited at a much shorter wavelength. In some embodiments, the binding of FITC to Taxol can be modified to reduce the Taxol's affinity to tubulin. Application of quantum dot (replacing traditional fluorescence photodonor) may produce more efficient and readily detectable FRET in this and other assays.

The previous description of the disclosed embodiments is provided to enable any person skilled in the art to make or use the biosensing devices, methods and systems. Various modifications to these embodiments will be readily apparent to those skilled in the art, and the generic principles defined herein may be applied to other embodiments without departing from the spirit or scope of the biosensing devices, methods and systems. Thus, the biosensing devices, methods and systems arenot intended to be limited to the embodiments shown herein but is to be accorded the widest scope consistent with the principles and novel features disclosed herein.

1. A device for detecting an analyte from within a patient's body, comprising:
a) an optical fiber having:
a first end; and
a second end configured to intermittently connect and disconnect to an analyzer;
and
b) a biosensing material attached to the first end comprising:
a polymer matrix;
at least one receptor molecule attached to the polymer matrix and labeled with a first detector molecule; and
at least one competitive binding molecule attached to the polymer matrix and labeled with a second detector molecule, wherein the at least one receptor molecule is capable of interacting with the at least one attached competitive binding molecule and with at least one analyte from the patient's body.

2. The device of claim 1, wherein the polymer matrix comprises polyethylene glycol.

3. The device of claim 1, wherein the at least one receptor molecule comprises a lectin.

4. The device of claim 3, wherein the at least one receptor molecule comprises concanavalin A.

5. The device of claim 1, wherein the at least one competitive binding analyte molecule comprises a polysaccharide.

6. The device of claim 5, wherein the polysaccharide comprises beta-cyclodextrin.

7. The device of claim 1, wherein the first and/or second detector molecule comprises a fluorophore.

8. The device of claim 7, wherein the first fluorophore comprises fluorescein isothiocyanate.

9. The device of claim 7, wherein the second fluorophore comprises tetramethylrhodamine isothiocyanate.

10. The device of claim 7, wherein the second fluorophore comprises a fluorescent semiconductor nanocrystal.

11. The device of claim 1, wherein the concentrations of the first and second detector molecules are low enough to minimize random proximity during FRET quenching, yet high enough to be detected by the analyzer.

12. The device of claim 7, wherein the size of the receptor and competitive binding molecule are not substantially larger than the Forster radius.

13. The device of claim 1, wherein the first end of the optical fiber further comprises a chemically altered adhesion region to which the biosensing material is attached.

14. The device of claim 1, wherein the first end of the optical fiber further comprises a mechanically altered adhesion region to which the biosensing material is attached.

15. A system for detecting an analyte from within a patient's body, comprising:
a) an optical fiber comprising:
a first end; and
a second end configured to intermittently connect and disconnect to an analyzer;
b) a biosensing material attached to the first end comprising:
a polymer matrix;
at least one receptor molecule attached to the polymer matrix and labeled with a first detector molecule; and
at least one competitive binding molecule attached to the polymer matrix and labeled with a second detector molecule, wherein the at least one receptor molecule is capable of interacting with the at least one attached competitive binding molecule and with at least one analyte from the patient's body;
and
c) an analyzer that is configured to rapidly and intermittently connect and disconnect to the second end of the optical fiber, and that is configured to emit light into the optical fiber, receive light from the biosensing material, and process information from the received light.

16. The system of claim 15, wherein the polymer matrix comprises polyethylene glycol.

17. The system of claim 15, wherein the at least one receptor molecule comprises a lectin.

18. The system of claim 17, wherein the at least one receptor molecule comprises concanavalin A.

19. The system of claim 15, wherein the at least one attached competitive binding molecule comprises a polysaccharide.

20. The system of claim 19, wherein the polysaccharide comprises beta-cyclodextrin.

21. The system of claim 15, wherein the first and/or second detector molecule comprises a fluorophore.

22. The system of claim 21, wherein the first fluorophore comprises fluorescein isothiocyanate.

23. The system of claim 21, wherein the second fluorophore comprises tetramethylrhodamine isothiocyanate.

24. The system of claim 21, wherein the second fluorophore comprises a fluorescent semiconductor nanocrystal.

25. The system of claim 15, wherein the concentrations of the first and second detector molecules are low enough to minimize random proximity during FRET quenching, yet high enough to be detected by the analyzer.

26. The system of claim 21, wherein the size of the receptor and analyte are not substantially larger than the Forster radius.

27. The system of claim 15, wherein the first end of the optical fiber further comprises a chemically altered adhesion region to which the biosensing material is attached.

28. The system of claim 15, wherein the first end of the optical fiber further comprises a mechanically altered adhesion region to which the biosensing material is attached.

29. The system of claim 15, wherein the analyzer is configured to automatically emit, receive, and analyze light upon insertion of the second end of the optical fiber.

30. The system of claim 15, wherein the analyzer further comprises a photodetector.

31. The system of claim 15, wherein the analyzer further comprises a tunable optical filter.

32. The system of claim 15, wherein the analyzer further comprises a user interface.

33. The system of claim 15, wherein the analyzer further comprises a substantially cone-shaped connecter by which the second end of the optical fiber connects to the analyzer.

34. A method of manufacturing an implantable biosensing device, comprising:
a) modifying the surface of a first end of an optical fiber to create an adhesion region;
b) submerging the first end of the optical fiber into a matrix precursor solution;
c) delivering ultraviolet light through a second end of the optical fiber; and
d) removing the first end of the optical fiber from the matrix precursor solution.

35. The method of claim 34, wherein the modifying step comprises immersing the first end of the optical fiber in an acidic solution to create an etched adhesion region.

36. The method of claim 35, wherein the modifying step further comprises cleaving off the end of the etched adhesion region.

37. The method of claim 34, further comprising repeating steps b) and d).

38. The method of claim 34, further comprising the step of applying ultraviolet light to the first end of the optical fiber.

39. The method of claim 34, wherein the modifying step comprises applying at least one chemical agent to the first end of the optical fiber to create an etched adhesion region.

40. The method of claim 38, wherein the at least one chemical agent comprises triethoxysilane.

41. The method of claim 34, wherein the modifying step comprises providing mechanical abrasion to the first end of the optical fiber to create an etched adhesion region.

42. The method of claim 34, wherein the matrix precursor solution comprises:
a) a polymer matrix;
b) at least one receptor molecule attached to the polymer matrix and labeled with a first detector molecule; and
c) at least one competitive binding molecule attached to the polymer matrix and labeled with a second detector molecule, wherein the at least one receptor molecule is capable of interacting with the at least one attached competitive binding molecules and with at least one analyte from the patient's body.

43. A method of detecting an analyte from within a patient's body, comprising:
a) implanting an optical fiber having an implanted and free end within the patient's body such that the implanted end lies within the percutaneous region and the free end protrudes from the patient's body;
b) allowing the implanted end of the optical fiber to remain in the percutaneous region of the body without removal for at least seven days;
c) allowing the free end of the optical fiber to remain unconnected to any device for a substantial portion of the at least seven days while the implanted end remains in the percutaenous region of the body;
d) connecting and disconnecting the free end to a measuring instrument; and
e) testing the analyte with the measuring instrument while it is connected to the free end;
wherein the optical fiber comprises:
a) a polymer matrix;
b) at least one receptor molecule attached to the polymer matrix and labeled with a first detector molecule; and
c) at least one competitive binding molecule attached to the polymer matrix and labeled with a second detector molecule, wherein the at least one receptor molecule is capable of interacting with the at least one attached competitive binding molecules and with at least one analyte from the patient's body.

44. The method of claim 43, wherein the polymer matrix comprises polyethylene glycol.

45. The method of claim 43, wherein the at least one receptor molecule comprises a lectin.

46. The method of claim 45, wherein the at least one receptor molecule comprises concanavalin A.

47. The method of claim 43 wherein the at least one attached competitive binding molecule comprises a polysaccharide.

48. The method of claim 47, wherein the polysaccharide comprises beta-cyclodextrin.

49. The method of claim 43, wherein the first and/or second detector molecule comprises a fluorophore.

50. The method of claim 49, wherein the first fluorophore comprises fluorescein isothiocyanate.

51. The method of claim 49, wherein the second fluorophore comprises tetramethylrhodamine isothiocyanate.

52. The method of claim 49, wherein the second fluorophore comprises a fluorescent semiconductor nanocrystal.

53. The method of claim 43, wherein the concentrations of the first and second detector molecules are low enough to minimize random proximity during FRET quenching, yet high enough to be detected by the analyzer.

54. The method of claim 49, wherein the size of the receptor and analyte are not substantially larger than the Forster radius.

55. The method of claim 43, wherein the optical fiber further comprises a chemically altered adhesion region to which the biosensing material is attached.

56. The method of claim 43, wherein the optical fiber further comprises a mechanically altered adhesion region to which the biosensing material is attached.
